(19) United States
(12) Patent Application Publication
(10) Pub. No.: US 2005/0158405 A1
(43) Pub. Date: Jul. 21, 2005

(54) Personal decontaminant

(76) Inventor: Alex Boukas, E. Setauket, NY (US)

Correspondence Address: Bodner & O'Rourke, LLP, 425 Broadhollow Road, Melville, NY, 11747, US

(21) Appl. No.: 11/041,009

(22) Filed: Jan. 21, 2005

Related U.S. Application Data
(60) Provisional application No. 60/537,905, filed on Jan. 21, 2004.

(51) Int. Cl. A61K 35/78; A61K 31/045

(52) U.S. Cl. 424/725; 514/724

(57) ABSTRACT

A composition suitable as a decontaminant is disclosed. The composition is a mixture of one or more alcohol compounds and one or more essential oils containing germicidal properties. The essential oil has concentration in a range of about 0.01% to 100% by volume of the composition with the remainder an alcohol containing compound.

This is a conversion of Provisional Patent Application Ser. No. 60/537,905 filed Jan. 21, 2004, the disclosures of which are incorporated herein by reference.

### FIELD OF INVENTION

The present invention relates to a new and improved devices for the dispensing of a personal decontaminant. The present invention also includes novel antimicrobial compositions The decontaminant can contain an antiseptic such as an alcohol, including, but not limited to an ethanol or isopropyl alcohol or can include one or more plant extracts including herbs with germicidal/disinfectant properties such as the following examples. Tea Tree Oil (Melaleuca Allernifolia), cedar, chamomile, eucalyptus, oatmeal, juniper, thyme (Thymus Vulgaris), bayberry, orange extract, grapefruit extract, aloe vera and others. The composition may also include blends of one or more alcohols and one or more herbal materials that have germicidal properties.

### BACKGROUND OF THE INVENTION

Bacterial infections are caused by the presence and growth of microorganisms that damage host tissue. The extent of infection is generally determined by how many organisms are present and the toxins they release. Worldwide, bacterial infections are responsible for more deaths than any other cause. Symptoms can include inflammation and swelling, pain, heat, redness, and loss of function. The most important risk factors are burns, severe trauma, low white blood cell counts, very old or young patients, patients on immunotherapy treatment, and anyone with malnutrition or vitamin deficiency.

Bacteria are generally spread from an already infected person to the newly infected person. The most common invasion routes are inhalation of airborne bacteria, ingestion into the stomach from dirty hands or utensils, or through contaminated food or water, direct contact with an infected area of another person's body, contaminated blood, and by insect bite.

Bacteria consist of only a single cell, but their small size is no indication of their complexity. Bacteria have been found to live in temperatures above the boiling point and in cold that would freeze your blood. They “eat” everything from sugar and starch to sunlight, sulfur and iron.

There are thousands of species of bacteria, but all of them are basically one of three different shapes. Some are rod- or stick-shaped and called bacilli. Others are shaped like little balls and called cocci. Others still are helical or spiral in shape, like the Borrelia pictured at the top of this page. Some bacterial cells exist as individuals while others cluster together to form pairs, chains, squares or other groupings.

The thousands of species of bacteria are responsible for causing Tuberculosis, bacterial sexually transmitted diseases, ear infections, bacterial meningitis, pneumonia, strep throat, eye infections, skin rashes and many more. In a mobile society, such as we live in, individuals are exposed to thousands of bacteria and these diseases everyday either by accident from coming in contact with other people, negligently by coming in contact with bacteria, or even by the intentional spread of contagious diseases by terrorists.

Viral infections are caused by viruses who depend on the host cells that they infect to reproduce. When found outside of host cells, viruses exist as a protein coat or capsid, sometimes enclosed within a membrane. The capsid encloses either DNA or RNA which codes for the virus elements. While in this form outside the cell, the virus is metabolically inert.

When it comes into contact with a host cell, a virus can insert its genetic material into its host, literally taking over the host's functions. An infected cell produces more viral protein and genetic material instead of its usual products. Some viruses may remain dormant inside host cells for long periods, causing no obvious change in their host cells, a stage known as the lysogenic phase. But when a dormant virus is stimulated, it enters the lytic phase: new viruses are formed, self-assemble, and burst out of the host cell, killing the cell and going on to infect other cells.

Viruses cause a number of diseases in humans, such as smallpox, the common cold, chickenpox, influenza, shingles, herpes, polio, rabies, Ebola, hanta fever, and AIDS.

Flu is short for “influenza”. The name goes back hundreds of years when the disease was thought to be caused by supernatural “influences”. There are several different viruses and bacteria which may infect the lung, but true flu is caused by orthomyxoviruses, of which there are three types, designated A, B, and C. Influenza C infects most people when they are young and rarely causes serious illness. Type B occasionally causes local outbreaks of flu and is usually confined to youngsters. Influenza A an important concern to mankind as this is the type of virus that has caused worldwide pandemic.

An influenza virion has about 500 “spikes” sticking out from its lipid envelope. About 80% of the spikes are a viral protein called hemagglutinin (HA). HA is influenza's receptor-binding protein. It plays the critical role of attaching the virus to the host cell. The other 20% of the spikes are a viral protein called neuraminidase, often abbreviated NA. This protein is an enzyme that destroys a host cell molecule called neuraminic or sialic acid. NA might play a part in getting the virus into the cell but its most important function is that it helps the newly made influenza virions to easily escape from the host cell so they can infect others.

In summary, HA is influenza's receptor-binding protein that helps the virion get into host cells and NA is an enzyme that helps the offspring virions to get out. Together, these two viral proteins are primarily responsible for the viruses' ability to cause disease, a property known as the virus' virulence.

Orthomyxoviruses usually infect the upper respiratory tract because these tissues have plenty of the receptors for the influenza virus. However, any mucous membrane will suffice as a point of entry. A common way to pick up the flu virus is to rub the moist corners of the eyes, nose or mouth after having shaken hands with someone who is shedding virus. The transmission of the flu presents a need for a discrete way to sanitize one's person.

The Center of Disease Control recommends as prevention against influenza washing your hands often with a hand hygiene product containing ethyl alcohol and emollients in a gel or wipe to clean and moisturize your hands. At this time, hand washing remains the most effective way to prevent the spread of infection. Frequent hand washing, especially during cold and flu season, will protect your health and the health of others.

Experimentation in the art has indicated the rapid bactericidal action of alcohols. Vegetative bacteria (e.g. Staphylococcus aureus, Streptococcus pyogenes, E. coli, Pseudomonas aeruginosa, Salmonella typhus, Serratia marscesens) are killed within 15 seconds of exposure. These same organisms are killed on surgical instruments contaminated with blood and purulent material within 30-60 seconds, with rare exposure of 2 minutes. Alcohol is most virucidal for the lipid enveloped viruses such as hepatitis and HIV (See Sterilization of Instruments & Gloves for Survival.www.uoguelph.ca/research/acs)

One of the problems with prior art uses of alcohol as a disinfectant is that the material to be disinfected typically has to be soaked in the alcohol in order for there to be adequate disinfection. According to the APIC (Association for Professionals in Infection Control and Epidemiology), ethyl alcohol and isopropyl alcohol are not effective in sterilizing instruments because they lack sporicidal activity and can't penetrate protein-rich materials. Isopropyl alcohol also lacks the ability to kill hydrophilic viruses. For these reasons, alcohol is classified as an intermediate level disinfectant.

Tea tree oil is a strong herbal disinfectant. Tea tree oil posses pronounced germicidal activity due to the terpinen-4-ol. Tea tree oil was first researched in 1923 in Australia. Clinical trials, mainly in Australia, have shown that tea tree is effective at treating a broad range of infectious conditions, notably fungal and skin problems such as vaginal yeast infections, acne, and warts. One of the most important constituents is known to be terpinen-4-ol, which is significantly antiseptic and well tolerated by the skin. The oil also contains cineol, which can irritate the skin. The cineol content varies—poor-quality oil has more than 10%; in some cases up to 65%. It is important that the commercial product not be derived from other Melaleuca species, some which contain high concentrations of cineole, a skin irritant that also reduces the antiseptic effectiveness of terpinen-4-ol. A recent study found the oil effective in vitro against methicillin-resistant Staphylococcus aureus strains. Although terpinen-4-ol is considered the primary active component, Australian researchers have confirmed that other components in the essential oil, including alpha-terpineol and linalool also contribute to its antimicrobial action (See herbs200.com retrieved Oct. 2, 2004).

Because of the risk individuals are exposed to everyday, there exists a need for an easy to use personal decontaminant that can be used daily to protect oneself from the transmission of diseases. A personal decontaminant can be brought everywhere and used in public facilities, on the subway, in airports, airplanes, restaurants, in office building, hotels, schools and many more places. A personal decontaminant becomes very important when traveling either domestic and especially internationally with the transfer of bacteria from unknown sources and exposure for the first time to certain foreign bacteria. Furthermore, not only is there a risk for humans but there is also a risk of exposure for animals. The present invention can also be used for pets such as dogs and cats when they come in the house from being outside with other animals.

Antibacterial personal care compositions are known in the art. Especially useful are antibacterial cleansing compositions, which typically are used to cleanse the skin and to destroy bacteria and other microorganisms present on the skin, especially the hands, arms, and face of the user. These compositions are frequently harsh to the skin and must be rinsed away with water to remove traces of the material from the skin. In addition, many of these materials are not suitable for use on clothing as they may have a tendency to stain.

Antibacterial compositions are used, for example, in the health care industry, food service industry, meat processing industry, and in the private sector by individual consumers. The widespread use of antibacterial compositions indicates the importance consumers place on controlling bacteria and other microorganism populations on skin. It is important, however, that antibacterial compositions provide a substantial and broad spectrum reduction in microorganism populations quickly and without problems associated with toxicity and skin irritation.

Thus, there exists a need for a personal decontaminant which is suitable for a quick, easy, and discrete way of decontaminating ones person using various dispensing mechanisms.

### SUMMARY OF INVENTION

Many natural environmentally friendly and non-toxic ingredients can be used, such as ethanol and/or isopropyl alcohol either alone or combined with one or more herbs as a personal decontaminant. These herbs can include tea tree oil (Melaleuca Allernifolia), cedar, chamomile, eucalyptus, oatmeal, juniper, thyme (Thymus Vulgaris), bayberry, orange extract, grapefruit extract, aloe vera and others.

The present invention is directed to a personal decontaminant in the form of a liquid, gel or spray solution that can be carried by an individual and readily dispensed to decontaminate bacteria by means of an applicator, towelette, sponge, cloth or other suitable dispensing means. The applicator can be a spray such as an aerosol spray that may be pumped to apply the decontaminent or a pressurized aerosol spray contain in, for example, a can. The personal decontaminent of the present invention is preferably contained in a container that can be readily carried in a purse or pocket and applied as necessary to the skin, clothes or other surfaces.

A personal decontaminant composition useful in the present invention can have from about 0.01 parts to 100 parts of an alcohol, such as ethanol, m-propyl and isopropanol, preferably 20 parts to 80 parts, and more preferably 40 parts to 70 parts.

In other embodiments there can be other alcohols such as terpinen-4-ol, other terpineols, linalool, cineol and the alcohols of monoterpenes and sesquiterpenes. Other suitable materials include orange, lemon, lime, as well as grapefruit seed extracts and tocopherols.

Additional ingredients besides water can include such plant extracts as garlic, onion, cloves, thyme, cinnamon, black and/or white pepper, capsicum, oregano, rosemary, allspice, basil, aniseed, fennel, caraway, nutmeg, sage, bay, coriander, turmeric, ginger, mint, eucalyptus, aloe, vanilla, mustard and horseradish. Also, the composition may include cocoa, tea, coffee, honey, legume extracts, radish and tomato and combinations of one or more of the foregoing materials.

The composition may also include iodine and/or chlorhexidine either alone or combined with other materials.

The composition may also contain one or more inert materials such as fillers, emulsifiers, preservatives, etc.

### OBJECTS OF THE INVENTION

One object of the invention is to provide a decontaminant composition in the form of a liquid that can be dispensed in an aerosol container.

Another embodiment of the invention is to provide a decontaminant composition in the form that can be dispensed in a pump spray.

Another object of the invention is to provide a decontaminant composition in the form that can be dispensed using a canister with a “bag in a can.

Another object of the invention is to provide a decontaminant composition in the form that can be dispensed in a canister in which the decontaminant is contained in a bag that is inserted into the canister and dispensed in to the form of a misted spray.

A further object of the invention is to provide a decontaminant composition in a form that can dispensed as a spray gel.

A still further object of the invention is to provide a decontaminant composition in a form that is contained in a canister of an applicator and applied with the roller ball.

Another object of the invention is to provide a decontaminant composition in a form that can be applied with bottle moistener, so that the decontaminant is contained in the bottle and is applied with a sponge attached to the top of the bottle or a sponge that is separate from the bottle.

A still further object of the invention is to provide a decontaminant composition in the form of replaceable cartridges that can be inserted in an applicator.

Another object of the invention is to provide a decontaminant composition in the form of a liquid solution which can be applied topically by any suitable means.

Another object of the invention is to provide a decontaminant composition with an alcohol base that can be used with a combination of natural ingredients such as herbs, food extracts and the like.

Another object of the invention is to provide a decontaminant composition with an alcohol base that can be used with a combination of natural ingredients such as herbs to be used as a personal use product, such as skin cleanser, a body splash, a wound care agent, a hand sanitizer, soap, a hard surface sanitizer, pet care products and the like.

Another object of the invention is to provide a decontaminant composition that will be effective in destroying harmful bacteria and be safe to use on the skin.

Another object of the invention is to provide a decontaminant composition that will be effective in destroying harmful bacteria and can be used for pets.

A still further object is to provide a decontaminant composition that has an antimicrobial.

### BRIEF DESCRIPTION OF THE DRAWINGS

FIGS. 1 and 2 depict a longitudinal cross section of spray device of an aerosol can containing the personal decontaminant.

FIG. 3 is a side view of an embodiment of a roll-on applicator containing the personal decontaminant.

FIG. 4 is a side view of the applicator of FIG. 3 with a cap engaged on the applicator to cover an application member of the applicator.

FIG. 5 is a side view of an embodiment of a pump spray applicator containing the personal decontaminant.

FIG. 6 is a frontal view of another embodiment of the personal decontaminant.

### DETAILED DESCRIPTION OF THE DRAWINGS

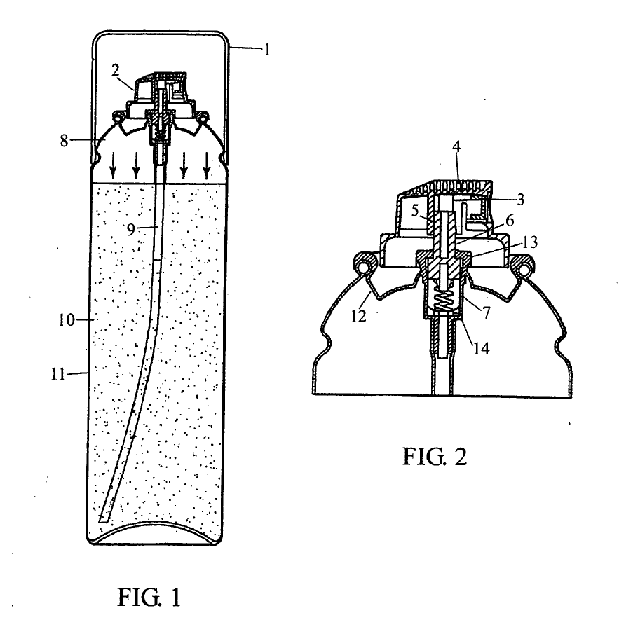

The personal decontaminant, represented by FIGS. 1 and 2 includes a container 11, containing a decontaminant 10 and a propellent gas mixture 8, a valve composed of a valve body 7, a dish 12, a rod for actuating the valve and ejecting the composition, a seal 13 and a spring 14. The container 11 is surmounted by a removable cap 1; the means for atomizing is composed of a ditube 9 attached to a valve body 7 and a push-button 2, the push-button comprises a coupling 5 which fits onto the rod 6, distribution channel 4 and a nozzle 3.

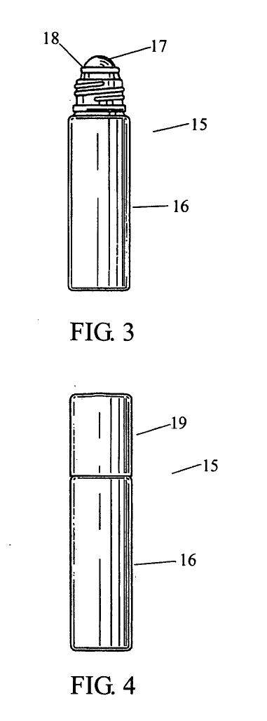

FIG. 3 shows an example of a roll-on applicator 15 comprising a container 16 and a rotatable application member 17 on the container 16. The container 16 contains the personal decontaminant.

The application member 17 may close an open end of the container 16. The application member 17 may be in flow communication with the composition contained in the container 12. The application member can have any shape, such as spherical, oval, regular, or irregular shape with a smooth or non-smooth service. The application member 17 may be retained on the container 16 so that the application member 17 is rotatably movable. For example, a retainer 18 may be provided to retain the application member 17 on the container 16 while permitting the rotation of the application member 17 during product application.

As shown in FIG. 4 the applicator 15 optionally has a removable cap 19 configured to cover the application member 14. The cap 19 may be releasably engaged with the container 15 in any known manner. For example, the cap 19 and the container 15 may have cooperating screw threading.

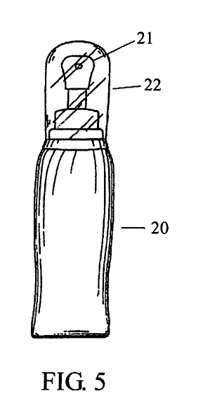

FIG. 5 shows the pump spray container 20 with the pump spray 21 dispensing the personal decontaminant and a cover 22 that prevents waste of the personal decontaminant.

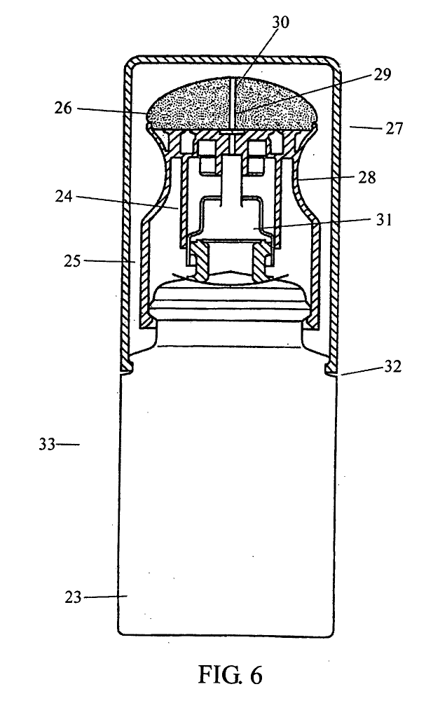

FIG. 6 is a container with a sponge top applicator. The personal decontaminant 33 comprises a hollow bodied container 23 and a dispenser portion 25. The dispenser portion 25 comprises an actuating mechanism 24. To operate, a user depresses an ergonomically shaped top portion 28, which then actuates a pump 31 and conduits 29 operate when the user presses the ergonomically shaped top portion thereby compressing a spring and a check valve creating a decrease in pressure within the hollow bodied container 23 and drawing up the personal decontaminant through the hollow bodied container 23 to the dispenser portion 25 by means of conduit 29 through the aperture 30. The conduit 29 meets the surface of the closed cell foam sponge tip 26 and deposits on the surface of the closed cell foam sponge tip 26. The user then glides the closed cell tip 26 across their skin to apply the personal decontaminant contained in the container 23. The personal decontaminant is protected with a removable cap 27.

While the present invention has been described with reference to the preferred embodiments, such embodiments are merely exemplary and are not intended to be limiting or represent an exhaustive enumeration of all aspects of the invention. It should be appreciated that the present invention is capable fo being embodied in other forms without departing from its essential characteristics.

### EXAMPLE 1

### Percentages as by Volume

| | |
|---|---|
| distilled water | 10-95% |
| ethanol | 0-90% |
| isopropyl alcohol | 0-90% |
| terpinen-4-01 | 0-90% |
| terpineol | 0-90% |
| linalool | 0-90% |
| cineol | 0-90% |
| monterpene alcohol | 0-90% |
| sesquiterpene alcohol | 0-90% |
| orange extract | 0-90% |
| lemon extract | 0-90% |
| lime extract | 0-90% |
| grapefruit seed extract | 0-90% |
| one or more tocopherols | 0-90% |

1. A composition suitable as a decontaminant, consisting essentially of:
a mixture of one or more alcohol compounds, said one or more alcohol compounds having a concentration in a range of about 0.01% to 100% by volume of the composition, and
a mixture one or more essential oils containing germicidal properties, said essential oil having concentration in a range of about 0.01% to 100% by volume of the composition.

2. An apparatus for applying a a decontaminant, comprising of:
a mixture of one or more alcohol compounds, said one or more alcohol compounds having a concentration in a range of about 0.01% to 100% by volume of the composition, and a mixture one or more essential oils containing germicidal properties, said essential oil having concentration in a range of about 0.01% to 100% by volume of the composition said apparatus comprising an applicator for applying said decontaminant to the skin or clothing of a user.
